(19) United States
(12) Patent Application Publication
(10) Pub. No.: US 2020/0222703 A1
(43) Pub. Date: Jul. 16, 2020

(54) WIRELESS IMPLANTABLE PULSE GENERATORS

(71) Applicant: Stimwave Technologies Incorporated, Pompano Beach, FL (US)

(72) Inventors: Laura Tyler Perryman, Pompano Beach, FL (US); Richard LeBaron, Miami Beach, FL (US); Chad David Andresen, Miami Beach, FL (US)

(21) Appl. No.: 16/691,771

(22) Filed: Nov. 22, 2019

Related U.S. Application Data
(60) Provisional application No. 62/790,875, filed on Jan. 10, 2019.

(51) Int. Cl. A61N 1/372; A61N 1/378; H02J 50/10; H02J 50/40; H02J 7/02

(52) U.S. Cl. CPC A61N 1/37235; A61N 1/37223; H02J 7/02; H02J 50/10; H02J 50/402; A61N 1/3787

(57) ABSTRACT

An implantable pulse generator includes a controller configured to generate a forward signal carrying electrical energy, a first antenna configured to send the forward signal to an implanted tissue stimulator such that the implanted tissue stimulator can use the electrical energy to generate one or more electrical pulses and deliver the one or more electrical pulses to a tissue, a communication module configured to receive instructions carried by an input signal from a programming module for generating the forward signal at the controller, and a second antenna configured to receive the input signal from the programming module.

### CROSS-REFERENCE TO RELATED APPLICATION

This application claims the benefit of U.S. Provisional Application No. 62/790,875, filed Jan. 10, 2019, and titled “Wireless Implantable Pulse Generators,” which is incorporated by reference.

### TECHNICAL FIELD

This disclosure relates to wireless, implantable pulse generators designed to power implanted tissue stimulators.

### BACKGROUND

Modulation of tissue within the body by electrical stimulation has become an important type of therapy for treating chronic, disabling conditions, such as chronic pain, problems of movement initiation and control, involuntary movements, dystonia, urinary and fecal incontinence, sexual difficulties, vascular insufficiency, and heart arrhythmia. For example, a pulse generator can be used to send electrical energy to electrodes on an implanted tissue stimulator that can pass pulsatile electrical currents of controllable frequency, pulse width, and amplitudes to a tissue.

### SUMMARY

In general, this disclosure relates to wireless implantable pulse generators designed to power implanted tissue stimulators. Such tissue stimulators are designed to deliver electrical therapy to surrounding tissues.

In one aspect, an implantable pulse generator includes a controller configured to generate a forward signal carrying electrical energy, a first antenna configured to send the forward signal to an implanted tissue stimulator such that the implanted tissue stimulator can use the electrical energy to generate one or more electrical pulses and deliver the one or more electrical pulses to a tissue, a communication module configured to receive instructions carried by an input signal from a programming module for generating the forward signal at the controller, and a second antenna configured to receive the input signal from the programming module.

Embodiments may provide one or more of the following features.

In some embodiments, the implantable pulse generator is a wireless pulse generator.

In some embodiments, the forward signal is an RF signal.

In some embodiments, the first antenna is configured to transmit signals having a frequency in a range of 300 MHz to 8 GHz.

In some embodiments, the first antenna is configured to transmit and receive energy via radiative coupling.

In some embodiments, the second antenna is configured to transmit signals having a frequency in range of 300 MHz to 8 GHz.

In some embodiments, the second antenna is configured to transmit and receive energy via inductive coupling.

In some embodiments, implantable pulse generator further includes a rechargeable battery for powering the implantable pulse generator.

In some embodiments, the second antenna is configured to transmit power to the rechargeable battery.

In some embodiments, the implantable pulse generator further includes a third antenna configured to transmit power to the rechargeable battery.

In some embodiments, the third antenna is configured to transmit signals having a frequency in a range of 300 MHz to 8 GHz.

In some embodiments, the third antenna is configured to transmit and receive energy via inductive coupling.

In some embodiments, the implantable pulse generator further includes a primary cell battery for powering the implantable pulse generator.

In some embodiments, the implantable pulse generator further includes one or more additional first antennas for communicating with one or more additional tissue stimulators.

In some embodiments, the implantable pulse generator further includes a housing that contains the controller, the first antenna, the second antenna, and the communication module.

In some embodiments, the housing is hermetically sealed.

In some embodiments, the housing is not hermetically sealed.

In some embodiments, the implantable pulse generator further include a power detector that can receive a reflected power signal from the implanted tissue stimulator via the first antenna.

In some embodiments, the controller is configured to adjust the forward signal based on the reflected power signal.

In some embodiments, the power detector includes an RF switch.

### DESCRIPTION OF DRAWINGS

FIG. 1 is a diagram of a tissue stimulation system. Components are not drawn to scale.

FIG. 2 is a block diagram of a programming module of the tissue stimulation system of FIG. 1.

FIG. 3 is a block diagram of a pulse generator of the tissue stimulation system of FIG. 1, including one antenna and a rechargeable battery.

FIG. 4 is a block diagram of a controller of the pulse generator of FIG. 3.

FIG. 5 is a block diagram of a power detector of the pulse generator of FIG. 3.

FIG. 6 is a block diagram of a tissue stimulator of the tissue stimulation system of FIG. 1.

FIG. 7 is a block diagram of a pulse generator that includes three antennas and a rechargeable battery.

FIG. 8 is a block diagram of a pulse generator that includes two antennas and a rechargeable battery.

FIG. 9 is a block diagram of a pulse generator that includes two antennas and a primary cell battery.

### DETAILED DESCRIPTION

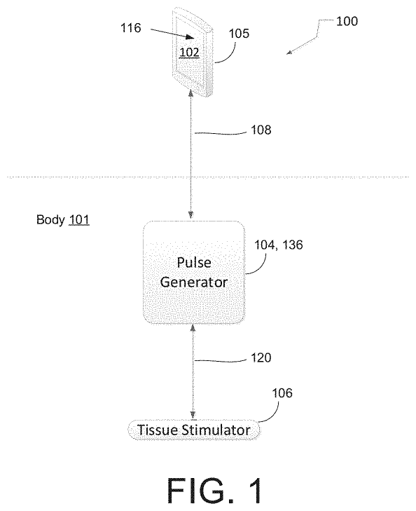

FIG. 1 illustrates a tissue stimulation system 100 designed to provide electrical therapy to a tissue (e.g., a neural tissue) within a body 101. In particular, the tissue stimulation system 100 is operable to send electrical pulses to the tissue to stimulate the tissue. Example tissues 101 that may be targeted by the tissue stimulation system 100 include nerve tissues in the spinal column, such as spinothalamic tracts, a dorsal horn, a dorsal root ganglia, dorsal roots, dorsal column fibers, and peripheral nerves bundles leaving a dorsal column or a brainstem. In some examples, the tissue may include one or more of cranial nerves, abdominal nerves, thoracic nerves, trigeminal ganglia nerves, nerve bundles of the cerebral cortex, nerve bundles of the deep brain, sensory nerves, and motor nerves. The tissue stimulation system 100 includes a programming module 102 implemented on a computing device 105, a pulse generator 104 that creates an electrical signal based on inputs received at the programming module 102, and a tissue stimulator 106 that generates electrical pulses based on instructions carried by the electrical signal.

The programming module 102 is a software application that enables a user (e.g., a patient, a technical representative, or a medical practitioner, such as a physician, a nurse, or another clinician) to view statuses (e.g., diagnostic statuses, equipment logs, localization of the tissue stimulator 106, and statuses of instructions sent to the tissue stimulator 106) of the pulse generator 104 and the tissue stimulator 106, set or change various operational parameters of the pulse generator 104 and the tissue stimulator 106 (e.g., a feedback sensitivity of the pulse generator 104 or RF power levels), and set or change stimulation parameters (e.g., an amplitude, stimulus pulse width, or stimulus pulse frequency) of the electrical pulses generated by the tissue stimulator 106. The software application is designed to support a wireless connection 108 (e.g., a radio frequency (RF) connection) between the computing device 105 and the pulse generator 106. Example computing devices 105 on which the programming module 102 may be implemented include a smart phone, a tablet or handheld computer, a laptop computer, a desktop computer, and other mobile and stationary computing devices.

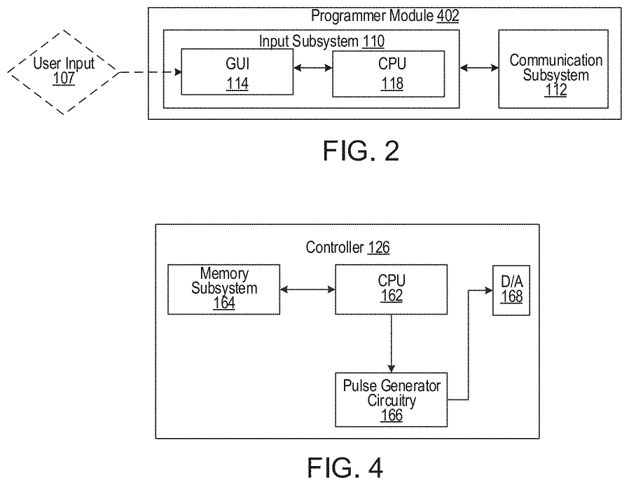

Referring to FIG. 2, the programming module 102 includes an input subsystem 110 by which the user can operate (e.g., view and control) the tissue stimulation system 100 and a communication subsystem 112 that can send signals (e.g., RF signals carrying instructions) to the pulse generator 104 via the wireless connection 108. Accordingly, the input subsystem 110 includes a graphical user interface (GUI) unit 114 that can generate one or more GUIs 116 by which the user can enter one or more inputs 107 on a touchscreen of the computing device 105 (e.g., or at a separate data entry device coupled to the computing device 105).

Example inputs 107 include system operation inputs, such as RF pulse rate, RF pulse width, and non-stimulus instructions for the implant (e.g., a localization mode or a self-diagnostics mode). Example inputs 107 also include stimulation inputs, such as pulse attributes (e.g., a pulse amplitude, a pulse frequency, and a pulse duration), as well as electrode polarization, electrode combinations (e.g., sources and sinks), an electrode setting of active or inactive, a total duration of the treatment, a pattern of the treatment. For example, therapy may include intermittent periods, pulse trains, and periodic iterations of pulse trains, mixed in with scheduled time with no stimulus pulses (e.g., 1 min, 5 min, etc. depending on the prescribed therapy). Therapy may also reflect electrode combinations (e.g., sources and sinks, an electrode setting of active or inactive, depending on the targeted nerves and placement/location of the electrodes, as well as the prescribed therapy). The inputs 107 may vary, depending on certain patient parameters, such as health, size, age, location of the tissue stimulator 106, depth of the tissue stimulator 106, tissue surrounding the stimulator Rx antenna and/or in the proximity of electrodes. For example, the pulse amplitude is typically set within a range of 0.1 mA to 30.0 mA, the pulse frequency is typically set within a range of 5 Hz to 50 kHz, and the pulse duration is typically set within a range of 5 μs to 2 ms.

While the tissue stimulation system 100 may be programmed with first inputs 107 during an initial surgical procedure in which the pulse generator 104 and the tissue stimulator 106 are implanted within the body 101, the inputs 107 can be adjusted later to account for a change in a patient's medical condition or body. In this manner, the tissue stimulation system 100 can continue to provide effective treatments over time. A clinician user may have the option of locking and/or hiding certain settings via one or more GUIs 116 to limit an ability of a patient user to view or adjust certain parameters that require detailed medical knowledge of neurophysiology, neuroanatomy, protocols for neural modulation, and safety limits of electrical stimulation.

The input subsystem 110 also includes a central processing unit (CPU) 118 for processing and storing data (e.g., including the one or more inputs 107) and for communicating with the communication subsystem 112. The communication subsystem 112 can transmit the RF signal (e.g., carrying instructions based on the one or more inputs 107, as well as other information) to the pulse generator 104 via the wireless connection 108. The communication subsystem 112 can also receive data (e.g., carried by an RF signal) from the pulse generator 104.

Referring again to FIG. 1, the pulse generator 104 is a wireless, implantable device that can receive instructions carried by an RF signal sent from the computing device 105 on which the programming module 102 is implemented. In some examples, the pulse generator 104 may be implanted subcutaneously at a distance of about 0.5 cm to about 12.0 cm from the site of the tissue stimulator 106. Because the pulse generator 104 is implantable within the body 103, the tissue stimulation system 100 may experience less loss of RF energy transmitted to the tissue stimulator 106, as compared to other implementations where a pulse generator is designed to be worn external to the body and therefore located further from a tissue stimulator.

The pulse generator 104 can generate a waveform based on the instructions and send a signal (e.g., an RF signal) carrying the waveform to the tissue stimulator 106 via a wireless connection 120 (e.g., an RF connection). The waveform encodes the attributes (e.g., the amplitude, the frequency, and the duration) of the pulses specified by the inputs 107. The signal also carries energy for powering the tissue stimulator 102. The pulse generator 104 can also receive a signal (e.g., an RF signal carrying feedback information) from the tissue stimulator 106. Accordingly, the pulse generator 104 includes microelectronics and other circuitry for generating, transmitting, and receiving such signals, as well as a housing 136 that contains these internal components.

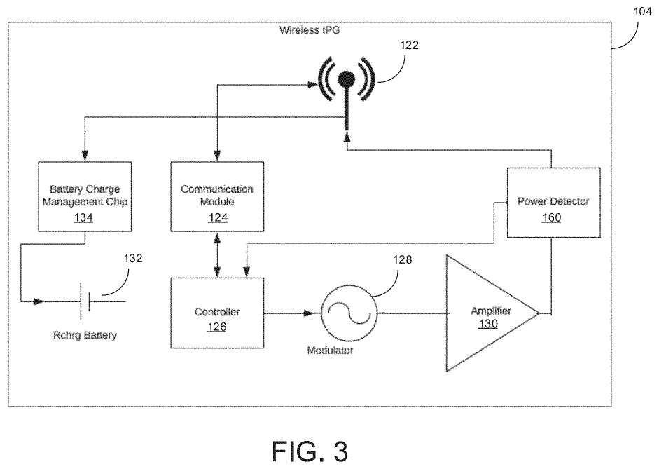

Referring to FIG. 3, the pulse generator 104 further includes an antenna 122 (e.g., a dipole antenna or any other small antenna or conductor configuration that can be used to receive RF power and/or communication and that fits within the dimensions of the pulse generator 104, such as a sub-wavelength patch antenna) that can receive a signal from the computing device 105 on which the programming module 102 is implemented. In addition to receiving signals from the computing device 105 carrying instructions for generating stimulus waveforms, the antenna 122 can also receive signals from the tissue stimulator 106 carrying feedback information related to the pulses actually delivered by the tissue stimulator 106 to the tissue. The antenna 122 can receive and send signals that have a frequency in a range of 300 MHz to 8 GHz.

The pulse generator 104 further includes a communication module 124 that relays instructions carried by the signal, a controller 126 that processes the instructions to generate a stimulus waveform, a modulator 128 that imparts a frequency in a range of 300 MHz to 8 GHz to the stimulus waveform, an amplifier 130 that imparts the inputted pulse amplitude on the stimulus waveform, and a power detector 160 that can process feedback information received from the tissue stimulator 106. In some implementations, the communication module 124 can execute a standard wireless communication protocol (e.g., Bluetooth, WiFi, or MICS). The amplifier 130 can send the modulated, amplified stimulus waveform to the antenna 122 for transmission to the tissue stimulator 106 and may operate via single stage or dual stage amplification. The pulse generator 104 also includes a battery 132 (e.g., a rechargeable battery) for powering the components of the pulse generator 104 and a battery charge management chip 134. The battery charge management chip 134 monitors a charge level of the battery 132 and uses energy carried by the signal sent from the antenna 122 to charge the battery 132 as needed.

In addition to the stimulus waveform carried by the signal transmitted from the antenna 122 to the tissue stimulator 106, the signal also provides an electric field within the body that can power the tissue stimulator 106 without the use of cables, such that the tissue stimulator 106 is a passive device that is coupled to the pulse generator 104 via electrical radiative coupling, as opposed to inductive coupling (e.g., via a magnetic field). As discussed above, the tissue stimulator 106 can generate an electrical pulse from the stimulus waveform and apply the electrical pulse to a target tissue in proximity to the tissue stimulator 106. In this context, the term electrical pulse refers to a phase of the stimulus waveform that directly produces stimulation of the tissue. Parameters of a charge-balancing phase of the stimulus waveform can also be controlled, as will be discussed in more detail below.

In some embodiments, the housing 136 of the pulse generator 104 is a hermetically sealed structure. In other embodiments, the housing 136 is not hermetically sealed, as the internal components of the pulse generator 104 may not be particularly susceptible to moisture. The housing 136 is typically made of one or more biocompatible materials that can protect the battery 132, but that still transmit radiation, such as titanium, silicon, polyurethane, stainless steel, and platinum-iridium, among others. The housing 136 is sized for placement within the body at locations such as subcutaneous space in the chest, abdomen, flank, buttock, thigh, or arm. Accordingly, the housing 136 typically has a length of about 5.0 cm to about 10.0 cm, a width of about 0.5 cm to about 5.0 cm, and a thickness of about 0.1 cm to about 2.0 cm. The housing 136 may have a generally rectangular, circular, or other cross-sectional shape.

Referring to FIG. 4, the controller 126 of the pulse generator includes a CPU 162 for handling data processing, a memory subsystem 164 (e.g., a local memory), pulse generator circuitry 166, and a digital/analog (D/A) converter 168. The controller 126 can control the stimulation parameters of the signal sent from the pulse generator 104 to the tissue stimulator 106. These stimulation parameter settings can affect the power, current level, and/or shape of the electrical pulses that will be applied by electrodes of the tissue stimulator 106, as will be discussed in more detail below. As discussed above, the stimulation parameters can be programmed by the user via the programming module 102 to set a repetition rate, a pulse width, an amplitude, and a waveform that will be transmitted by RF energy to a receive (RX) antenna within the tissue stimulator 106.

The controller 126 can store received parameter settings in the memory subsystem 164 until the parameter settings are modified by new input data received from the programmer module 102. The CPU 162 can use the stimulation parameters stored in the memory subsystem 164 to control the pulse generator circuitry 166 to generate a stimulus waveform that is modulated by the modulator 128 in a range of 300 MHz to 8 GHz. The resulting stimulus waveform may then be amplified by the amplifier 130 and sent through an RF switch of the power detector 160 to the antenna 122 to reach the RX antenna of the tissue stimulator through a depth of tissue.

In some examples, the RF signal sent by the antenna 122 may simply be a power transmission signal used by tissue stimulator 106 to generate electric pulses. In other examples, the RF signal sent by the antenna 122 may be a telemetry signal that provides instructions about various operations of the tissue stimulator 106. The telemetry signal may be sent by the modulation of the carrier signal through the skin. The telemetry signal is used to modulate the carrier signal (e.g., a high frequency signal) that is coupled to the antenna 122 and does not interfere with the input for powering the tissue stimulator 106 received at the same RX antenna of the tissue stimulator 106. In some embodiments, the telemetry signal and the power transmission signal are combined into one signal, where the RF telemetry signal is used to modulate the power transmission signal such that the tissue stimulator 106 is powered directly by the telemetry signal. Separate subsystems in the tissue stimulator 106 harness power contained in the telemetry signal and interpret data content of the telemetry signal, as will be discussed in more detail below.

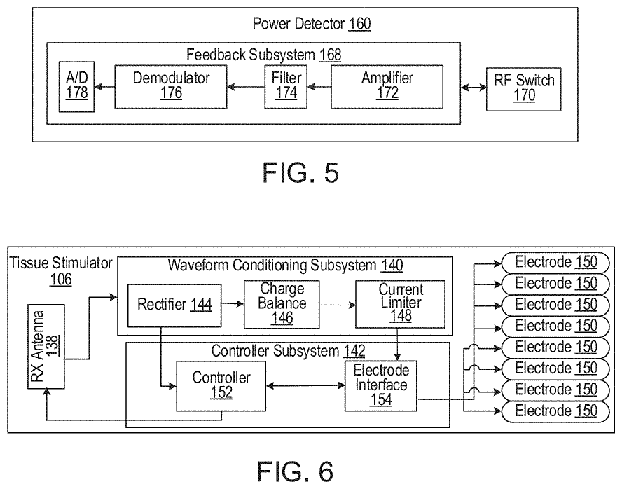

Referring to FIG. 5, the power detector 160 includes a feedback subsystem 168 and an RF switch 170. The feedback subsystem 168 includes reception circuitry for receiving and extracting telemetry or other feedback signals from tissue stimulator 106 and/or reflected RF energy from the signal sent by antenna 122. The feedback subsystem 168 includes an amplifier 172, a filter 174, a demodulator 176, and an A/D converter 178. The feedback subsystem 168 receives a forward power signal and converts this high-frequency AC signal to a DC level that can be sampled and sent to the controller 126. In this way, the characteristics of the generated RF pulse can be compared to a reference signal within the controller 126. If a disparity (e.g., a computed error) exists in any parameter, the controller 126 can adjust the output. In some examples, the value of the adjustment is proportional to the disparity. The controller 126 can also apply additional inputs and limits on the adjustment, such as a signal amplitude of a reverse power signal received from the tissue stimulator 106 and any predetermined maximum or minimum values for various pulse parameters.

The reverse power signal can be used to detect fault conditions in the pulse generator 104. For an ideal condition, when the antenna 122 has an impedance that is perfectly matched to that of the tissue that it contacts, the electromagnetic waves generated from the pulse generator 104 pass unimpeded from the antenna 122 into the body tissue. However, in real-world situations, a large degree of variability exists in the body types of users, types of clothing worn, and positioning of the antenna 122 relative to the body surface. Since the impedance of the antenna 122 depends on the relative permittivity of the underlying tissue and any intervening materials and on an overall separation distance of the antenna 122 from the skin, there can be an impedance mismatch at the interface between the antenna 122 and the skin surface of the body. When such a mismatch occurs, electromagnetic waves sent from the pulse generator 104 are partially reflected at this interface, and this reflected energy propagates backward to the antenna 122.

The RF switch 170 may be a multipurpose device (e.g., a dual directional coupler) that passes the relatively high amplitude, extremely short duration RF pulse to the antenna 122 with minimal insertion loss, while simultaneously providing two low-level outputs to the feedback subsystem 168. One output delivers a forward power signal to the feedback subsystem 168, where the forward power signal is an attenuated version of the RF pulse sent to the antenna 122, and the other output delivers a reverse power signal to a different port of the feedback subsystem 168, where reverse power is an attenuated version of the reflected RF energy from the antenna 122.

During the on-cycle time (e.g., while an RF signal is being transmitted to tissue stimulator 106), the RF switch 170 is set to send the forward power signal to feedback subsystem 168. During the off-cycle time (e.g., while an RF signal is not being transmitted to the tissue stimulator 106), the RF switch 170 can switch to a receiving mode in which the reflected RF energy and/or RF signals from the tissue stimulator 106 are received to be analyzed in the feedback subsystem 168.

The RF switch 170 may prevent the reflected RF signal from propagating directly back into the amplifier 172 by attenuating the reflected RF signal and then sending the attenuated signal to the feedback subsystem 168. The feedback subsystem 168 can convert this high-frequency AC signal to a DC level that can be sampled and sent to the controller 126. The controller 126 can then calculate a reflected power ratio of the amplitude of the reverse power signal to the amplitude of the forward power signal. The reflected power ratio may indicate a severity of an impedance mismatch.

The controller 126 can measure the ratio in real time, and according to preset thresholds for this measurement, the controller 126 can modify the level of RF power generated by the pulse generator 104. For example, for a moderate degree of reflected power, the controller 126 may increase the amplitude of RF power sent to the antenna 122, as would be needed to compensate for slightly non-optimum, but an acceptable coupling of the antenna 122 to the body. For higher reflected power ratios, the controller 126 may prevent operation of the pulse generator 104 by setting a fault code that indicates that the antenna 122 has little or no coupling with the body. This type of reflected power fault condition can also be generated by a poor or broken connection to the antenna 122. In either case, it may be desirable to stop RF transmission when the reflected power ratio is above a defined threshold, because internally reflected power can lead to unwanted heating of internal components, and this fault condition means that the system cannot deliver sufficient power to the tissue stimulator 106 to deliver therapy to the patient.

Referring to FIG. 6, the tissue stimulator 106 includes an antenna 138 (e.g., a dipole antenna or a thin wire antenna), a waveform conditioning subsystem 140, a controller subsystem 142, and multiple electrodes 150. The tissue stimulator 106 may include two to sixteen electrodes 150. The antenna 138 can receive the RF signal sent from the pulse generator 104 via the wireless connection 120 and relay the stimulus waveform carried by the RF signal to the waveform conditioning subsystem 140. The waveform conditioning subsystem 140 can make the stimulus waveform suitable for pulse generation and accordingly includes a rectifier 144, a charge balance component 146, and a current limiter 148. The controller subsystem 142 can route a conditioned stimulus waveform to the electrodes 150 and accordingly includes a controller 152 and an electrode interface 154.

The rectifier 144 rectifies the RF signal received by the antenna 138 and sends a rectified signal to the charge balance component 146. The charge balance component 146 is configured to create one or more counter-acting electrical pulses to ensure that the one or more electrical pulses applied by the electrodes 150 have a net charge of substantially zero, such that the electrical pulses applied by the electrodes 150 to the tissue are charge-balanced. The charge-balanced electrical pulses are passed through the current limiter 148 to the controller subsystem 142. The current limiter 148 ensures that a current level of the electrical pulses sent to the electrodes 150 is not above a threshold current level. For example, an amplitude (e.g., a current level, a voltage level, or a power level) of the stimulus waveform received at the antenna 138 may directly determine the amplitude of the electrical pulses applied by the electrodes 150 to the tissue. The current limiter 148 can prevent an excessive current or charge from being applied by the electrodes 150. In some examples, the current limiter 148 may be used in other cases, such as preventing unsafe current levels and ensuring that stimulation amplitude meets the expected value.

Generally, for constant current stimulation pulses, pulses should be charge-balanced such that an amount of cathodic current equals an amount of anodic current, which is typically called biphasic stimulation. Charge density is the amount of current multiplied by a duration that the current is applied. Charge density is typically expressed in units of uC/cm². In order to avoid irreversible electrochemical reactions (e.g., a pH change, electrode dissolution, or tissue destruction), no net charge should appear at the electrode-electrolyte interface, and it is generally acceptable to have a charge density less than 30 uC/cm². Biphasic stimulating current pulses ensure that no net charge appears at the electrodes 150 after each stimulation cycle and that the electrochemical processes are balanced to prevent net dc currents. Thus, the tissue stimulator 106 is designed to ensure that the resulting stimulus waveform has a net zero charge. Charge balanced stimuli are thought to have minimal damaging effects on tissue by reducing or eliminating electrochemical reaction products created at an electrode-tissue interface.

As mentioned above, a stimulus pulse may have a negative voltage or current, called the cathodic phase of the waveform. Stimulating electrodes 150 may have both cathodic and anodic phases at different times during the stimulus cycle. An electrode 150 that delivers a negative current with sufficient amplitude to stimulate adjacent neural tissue may be referred to as a “stimulating electrode” 150. During the stimulus phase, the stimulating electrode 150 acts as a current sink. One or more additional electrodes 150 act as a current source and may be referred to as “return electrodes” 150. Return electrodes 150 are positioned elsewhere in the tissue at some distance from the stimulating electrodes 150. When a typical negative stimulus phase is delivered to tissue at the stimulating electrode 150, the return electrode 150 has a positive stimulus phase. During the subsequent charge balancing phase, the polarities of each electrode 150 are reversed.

In some implementations, the charge balance component 146 uses one or more blocking capacitors placed electrically in series with the stimulating electrodes 150 and body tissue at a location between the point of stimulus generation within the stimulator circuitry and the point of stimulus delivery to tissue to form a resistor-capacitor (RC) network. In a multi-electrode stimulator, one charge-balance capacitor may be used for each electrode 150, or a centralized capacitor may be used within the stimulator circuitry prior to the point of electrode selection. The RC network can block direct current (DC). However, the RC network can also prevent low-frequency alternating current (AC) from passing to the tissue. The frequency below which the series RC network essentially blocks signals is commonly referred to as the cutoff frequency, and in some embodiments, the design of the tissue stimulation system 100 ensures that the cutoff frequency is not above the fundamental frequency of the stimulus waveform. For example, the tissue stimulator 106 may have a charge-balance capacitor with a value chosen according to the measured series resistance of the electrodes 150 and the tissue environment in which the tissue stimulator 106 is implanted. By selecting a specific capacitance value, the cutoff frequency of the RC network in this embodiment is at or below the fundamental frequency of the stimulus pulse.

In other implementations, the cutoff frequency may be chosen to be at or above the fundamental frequency of the stimulus such that the stimulus waveform (e.g., the drive waveform) created prior to the charge-balance capacitor may be non-stationary, where the envelope of the drive waveform is varied during the duration of the drive pulse. For example, in one embodiment, the initial amplitude of the drive waveform is set at an initial amplitude Vi, and the amplitude is increased during the duration of the pulse until it reaches a final value k*Vi. By changing the amplitude of the drive waveform over time, the shape of the stimulus waveform passed through the charge-balance capacitor is also modified. The shape of the stimulus waveform may be modified in this fashion to create a physiologically advantageous stimulus.

In some implementations, the tissue stimulator 106 may create a drive-waveform envelope that follows the envelope of the RF pulse received by the antenna 138. In this case, the pulse generator 104 can directly control the envelope of the drive waveform within the tissue stimulator 106, and thus no energy storage may be required inside of the tissue stimulator 106, itself. In this implementation, the stimulator circuitry may modify the envelope of the drive waveform or may pass it directly to the charge-balance capacitor and/or electrode-selection stage.

In some implementations, the tissue stimulator 106 may deliver a single-phase drive waveform to the charge balance capacitor or it may deliver multiphase drive waveforms. In the case of a single-phase drive waveform (e.g., a negative-going rectangular pulse), this pulse comprises the physiological stimulus phase, and the charge-balance capacitor is polarized (charged) during this phase. After the drive pulse is completed, the charge balancing function is performed solely by the passive discharge of the charge-balance capacitor, where is dissipates its charge through the tissue in an opposite polarity relative to the preceding stimulus. In one implementation, a resistor within the tissue stimulator 106 facilitates the discharge of the charge-balance capacitor. In some implementations, using a passive discharge phase, the capacitor may allow virtually complete discharge prior to the onset of the subsequent stimulus pulse.

In the case of multiphase drive waveforms, the tissue stimulator 106 may perform internal switching to pass negative-going or positive-going pulses (phases) to the charge-balance capacitor. These pulses may be delivered in any sequence and with varying amplitudes and waveform shapes to achieve a desired physiological effect. For example, the stimulus phase may be followed by an actively driven charge-balancing phase, and/or the stimulus phase may be preceded by an opposite phase. Preceding the stimulus with an opposite-polarity phase, for example, can have the advantage of reducing the amplitude of the stimulus phase required to excite tissue.

In some implementations, the amplitude and timing of stimulus and charge-balancing phases is controlled by the amplitude and timing of RF pulses from the pulse generator 104, and in other implementations, this control may be administered internally by circuitry onboard the tissue stimulator 106, such as the controller subsystem 142. In the case of onboard control, the amplitude and timing may be specified or modified by data commands delivered from the pulse generator 104.

Generally, for a given electrode 150 having several square millimeters of surface area, it is the charge per phase that should be limited, with regard to safety (e.g., where the charge delivered by a stimulus phase of the electrical pulse is the integral of the current). However, in some cases, a limit can instead be placed on the current, where the maximum current multiplied by the maximum possible pulse duration is less than or equal to the maximum safe charge. More generally, the current limiter 148 acts as a charge limiter that limits a characteristic (e.g., a current or a duration) of the electrical pulses so that the charge per phase remains below a threshold level (e.g., a safe charge limit).

In the event that the tissue stimulator 102 receives a “strong” pulse of RF power sufficient to generate a stimulus phase of the electrical pulse that would exceed the safe charge limit, the current limiter 148 can automatically limit or “clip” the stimulus phase to maintain the total charge of the stimulus phase within the safe charge limit. The current limiter 148 is a passive current limiting component that cuts the signal to the electrodes 150 once the safe current limit (e.g., a threshold current level) is reached. Alternatively, or additionally, the current limiter 148 may communicate with the electrode interface 154 of the controller subsystem 142 to turn off all of the electrodes 150 to prevent tissue-damaging current levels from being applied to the tissue.

Furthermore, such a clipping action may trigger a feedback control mode of the current limiter 148. For example, the clipping action may cause the controller 152 to send a threshold power data signal to the pulse generator 104 via the antenna 138 and the wireless connection 120. The power detector 160 of the pulse generator 104 detects the threshold power data signal and demodulates the signal into data that is communicated to the controller 126 of the pulse generator 104. In response to receiving the signal, the controller 126 may execute algorithms to reduce the RF power generated by the pulse generator 104 or may cut the RF power generated by the pulse generator 104 completely. In this manner, the pulse generator 104 can reduce the RF power delivered to the tissue if the tissue stimulator 106 reports receipt of excess RF power.

Alternatively to routing the rectified stimulus waveform to the charge balance 546, the rectifier 144 may route the rectified stimulus waveform to the controller 152 of the controller subsystem 142. The controller 152 can also communicate with the electrode interface 154 to control various aspects of setting up the electrodes 150 and electrical pulses routed to the electrodes 150. The electrode interface 154 may act as a multiplex and control a polarity and a switching of each of the electrodes 150. For instance, in some examples, multiple electrodes 150 of the tissue stimulator 106 are in contact with the tissue, and for a given electrical pulse, the pulse generator 104 can arbitrarily assign one or more electrodes 150 to act as a stimulating electrode 150, one or more electrodes 150 to act as a return electrode 150, or one or more electrodes 150 to be inactive. The assignments can be carried by the signal that carries the stimulus pulse parameters via the wireless connection 120. The controller 152 uses the assignments to set the electrode interface 154 accordingly. In some examples, it may be physiologically advantageous to assign one or two electrodes 150 as stimulating electrodes 150 and to assign all remaining electrodes 150 as return electrodes 150.

Furthermore, for a given electrical pulse, the controller 152 may control the electrode interface 154 to divide the current arbitrarily or divide the current among the designated stimulating electrodes 150 according to instructions from the pulse generator 104. Such control of the electrode assignment and control of the current can be advantageous since, in some examples, the electrodes 150 may be spatially distributed along various neural structures. Therefore, according to strategic designation of a stimulating electrode 154 at particular locations and proportioning of the current at the particular locations, the current distribution on the tissue can be modified to selectively activate specific neural targets. This strategy of current steering can improve a therapeutic effect of the treatment.

In some examples, a time course of electrical pulses may be arbitrarily manipulated. For example, a given stimulus waveform may be initiated at a time T_start and terminated at a time T final, and this time course may be synchronized across all stimulating and return electrodes 150. Furthermore, a frequency of repetition of the stimulus cycle may be synchronized for all of the electrodes 150. However, in some examples, the controller 152 (e.g., either on its own or according to instructions received from the pulse generator 104) can control the electrode interface 154 to designate one or more subsets of electrodes 150 to deliver stimulus waveforms with non-synchronized start and stop times and can arbitrarily and independently specify the frequency of repetition of each stimulus cycle.

For example, a tissue stimulator 106 having eight electrodes 150 may be configured to have a subset of five electrodes 150 (e.g., set A) and a subset of three electrodes 150 (e.g., set B). Set A may be configured to use two of its electrodes 150 as stimulating electrodes 150 and the remainder of its electrodes 150 as return electrodes 150. Set B may be configured to have just one stimulating electrode 150. The controller 152 could then specify that set A deliver a stimulus phase with 3 mA current for a duration of 200 us, followed by a charge-balancing phase that lasts 400 us. This stimulus cycle could be specified to repeat at a rate of 60 cycles per second. Then, for set B, the controller 152 could specify a stimulus phase with 1 mA current for duration of 500 us, followed by a charge-balancing phase that lasts 800 us. The repetition rate for the set B stimulus cycle can be set independently of repetition rate for set A (e.g., at 25 cycles per second). Or, in some examples, the controller 152 may match the repetition rates for set A and set B and specify relative start times of the stimulus cycles to be coincident in time or to be arbitrarily offset from one another by a delay interval.

In some examples, the controller 152 can arbitrarily shape the amplitude of the stimulus waveform, and in some cases, according to instructions received from the pulse generator 104. The stimulus phase may be delivered by a constant current source or a constant voltage source, and this type of control may generate characteristic waveforms that are static. For example, a constant current source can generate a characteristic rectangular pulse in which a current waveform has a very steep rise, a constant amplitude for a duration of the stimulus, and then a very steep return to a baseline. Alternatively, or additionally, the controller 152 can increase or decrease the level of current at any time during the stimulus phase and/or during the charge balancing phase. Thus, in some examples, the controller 152 can deliver arbitrarily shaped stimulus waveforms, such as a triangular pulse, sinusoidal pulse, or a Gaussian pulse. Similarly, the charge balancing phase can have an arbitrarily-shaped amplitude, and a leading anodic pulse (e.g., prior to the stimulus phase) may also be arbitrarily-shaped.

As discussed above, the pulse generator module 104 can remotely control stimulus parameters of the electrical pulses applied to the tissue by the electrodes 150 and monitor feedback from the tissue stimulator 106 based on RF signals received from the tissue stimulator 106. For example, a feedback detection algorithm implemented by the pulse generator 104 can monitor data sent wirelessly from the tissue stimulator 106, including information about the energy that the tissue stimulator 106 is receiving from the pulse generator 104 and information about the stimulus waveform being delivered to the electrodes 150. Accordingly, the circuit components internal to the tissue stimulator 106 may also include circuitry for communicating information back to the pulse generator module 104 to facilitate the feedback control mechanism. For example, the tissue stimulator 106 may send to the pulse generator 104 a stimulus feedback signal that is indicative of parameters of the electrical pulses, and the pulse generator 104 may employ the stimulus feedback signal to adjust parameters of the signal sent to the tissue stimulator 106.

The controller subsystem 142 may transmit informational signals, such as a telemetry signal, through the antenna 138 to communicate with the pulse generator 104 during its receive cycle. For example, the telemetry signal from the tissue stimulator 106 may be coupled to the modulated signal on the antenna 138, during the on and off state of the transistor circuit to enable or disable a waveform that produces the corresponding RF bursts necessary to transmit to the external (or remotely implanted) pulse generator 104. The antenna 138 may be connected to electrodes 150 in contact with the tissue to provide a return path for the transmitted signal. An A/D converter can be used to transfer stored data to a serialized pattern that can be transmitted on the pulse modulated signal from the antenna 138.

A telemetry signal from the tissue stimulator 106 may include stimulus parameters, such as the power or the amplitude of the current that is delivered to the tissue from the electrodes 150. The feedback signal can be transmitted to the pulse generator 104 to indicate the strength of the stimulus at the tissue by means of coupling the signal to the antenna 138, which radiates the telemetry signal to the pulse generator 104. The feedback signal can include either or both an analog and digital telemetry pulse modulated carrier signal. Data (e.g., stimulation pulse parameters and measured characteristics of stimulator performance) can be stored in an internal memory device within the tissue stimulator 106 and sent on the telemetry signal. The frequency of the carrier signal may be in a range of 300 MHz to 8 GHz.

In the feedback subsystem 168 of the power detector 160, the telemetry signal can be down modulated using the demodulator 176 and digitized by being processed through the A/D converter 178. The digital telemetry signal may then be routed to the CPU 162 of the controller 126 with embedded code, with the option to reprogram, to translate the signal into a corresponding current measurement in the tissue based on the amplitude of the received signal. The CPU 162 can compare the reported stimulus parameters to those held in memory subsystem 164 to verify that the tissue stimulator 106 delivered the specified stimuli to target nerve tissue. For example, if the tissue stimulator 106 reports a lower current than was specified, the power level from the pulse generator 104 can be increased so that the tissue stimulator 106 will have more available power for stimulation. The tissue stimulator 106 can generate telemetry data in real time (e.g., at a rate of 8 kbits per second). All feedback data received from the tissue stimulator 106 can be logged against time and sampled to be stored for retrieval to a remote monitoring system accessible by a health care professional for trending and statistical correlations.

The sequence of remotely programmable RF signals received by the antenna 138 may be conditioned into waveforms that are controlled within the tissue stimulator 106 by the controller subsystem 142 and routed to the appropriate electrodes 150 that are located in proximity to the target nerve tissue. For instance, the RF signal transmitted from the pulse generator 104 may be received by antenna 138 and processed by the waveform conditioning subsystem 140 to be converted into electrical pulses applied to the electrodes 150 through the electrode interface 154.

Thus, in order to provide an effective therapy for a given medical condition, the tissue stimulation system 100 can be tuned to provide the optimal amount of excitation or inhibition to the nerve fibers by electrical stimulation. A closed loop feedback control method can be used in which the output signals from the tissue stimulator 106 are monitored and used to determine the appropriate level of neural stimulation current for maintaining effective neuronal activation. Alternatively, in some cases, the patient can manually adjust the output signals in an open loop control method.

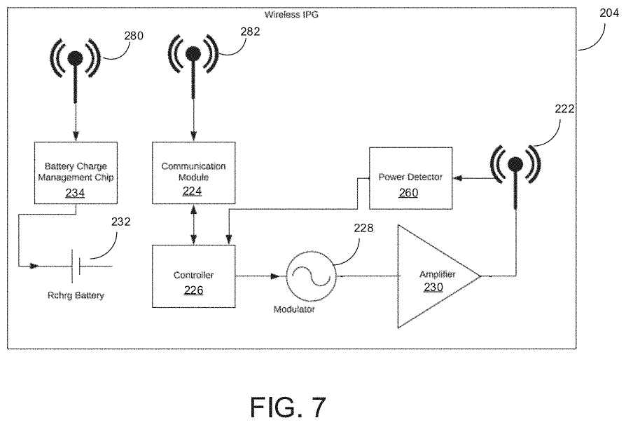

While the pulse generator 104 has been described and illustrated as including certain dimensions, sizes, shapes, materials, arrangements, and configurations, in some embodiments, tissue stimulation systems that are otherwise similar in structure and function to either of the tissue stimulation system 100 may include a pulse generator that has one or more of dimensions, sizes, shapes, materials, arrangements, and configurations that are different from those of the pulse generator 104. For example, a tissue stimulation system that is otherwise similar to the tissue stimulation system 100 may include a wireless, implantable pulse generator 204 that has a different configuration, as illustrated in FIG. 7. The pulse generator 204 is similar in structure and function to the pulse generator 104, except that the pulse generator 204 includes three antennas. For example, the pulse generator 204 includes a first antenna 222 by which the pulse generator 204 can communicate with the tissue stimulator 106 over a range of 300 MHz to 8 GHz Hz, a second antenna 280 by which a battery charge management chip 234 can communicate with a wireless charger over a low frequency range of 1 kHz to 5 MHz via inductive coupling, and a third antenna 282 by which the communication module 224 can communicate with the programming module 102 over a higher frequency range of 300 MHz to 8 GHz. Any of the antennas 222, 280, 282 may be a dipole antenna or a thin wire antenna.

The pulse generator 204 includes additional components that function substantially similarly to those described for the pulse generator 104. For example, the pulse generator 204 further includes a communication module 224 that relays instructions carried by the signal received from the programming module 102, a controller 226 that processes the instructions to generate a stimulus waveform, a modulator 228 that imparts a frequency in a range of 300 MHz to 8 GHz to the stimulus waveform, an amplifier 230 that imparts the inputted pulse amplitude on the stimulus waveform, and a power detector 260 that can process feedback information received from the tissue stimulator 106. The pulse generator 204 also includes a battery 232 (e.g., a rechargeable battery) for powering the components of the pulse generator 204.

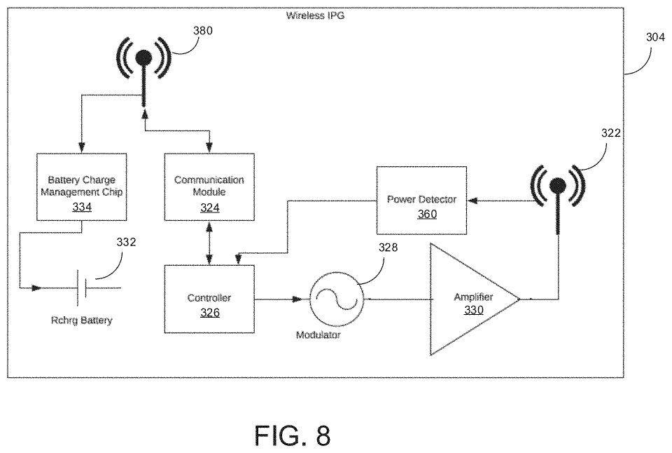

A tissue stimulation system that is otherwise similar to the tissue stimulation system 100 may include a wireless, implantable pulse generator 304 that has yet a different configuration, as illustrated in FIG. 8. The pulse generator 304 is similar in structure and function to the pulse generator 104, except that the pulse generator 304 includes two antennas. For example, the pulse generator 304 includes a first antenna 322 by which the pulse generator 304 can communicate with the tissue stimulator 106 over a range of 300 MHz to 8 GHz and a second antenna 380 by which a battery charge management chip 334 can communicate with a wireless charger over a low frequency range of 1 kHz to 5 MHz via inductive coupling and by which the communication module 324 can communicate with the programming module 102 over a higher frequency range of 300 MHz to 8 GHz. Either of the antennas 322, 380 may be a dipole antenna or a thin wire antenna.

The pulse generator 304 includes additional components that function substantially similarly to those described for the pulse generator 104. For example, the pulse generator 304 further includes a communication module 324 that relays instructions carried by the signal received from the programming module 102, a controller 326 that processes the instructions to generate a stimulus waveform, a modulator 328 that imparts a frequency in a range of 300 MHz to 8 GHz to the stimulus waveform, an amplifier 330 that imparts the inputted pulse amplitude on the stimulus waveform, and a power detector 360 that can process feedback information received from the tissue stimulator 106. The pulse generator 304 also includes a battery 332 (e.g., a rechargeable battery) for powering the components of the pulse generator 304.

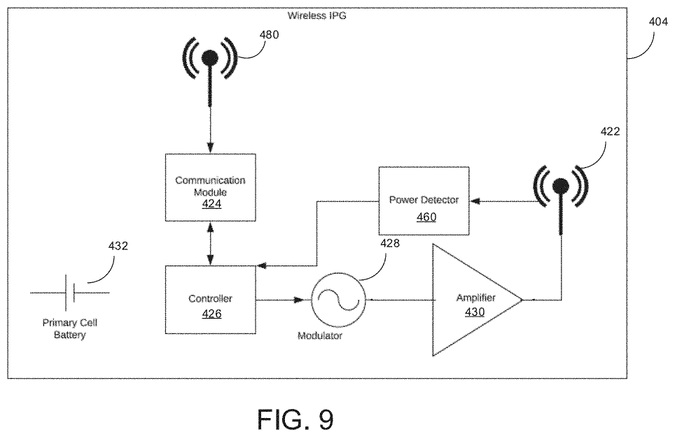

In some embodiments, a tissue stimulation system that is otherwise similar to the tissue stimulation system 100 may not include a rechargeable battery, as illustrated in FIG. 9. For example, a wireless, implantable pulse generator 404 is similar in structure and function to the pulse generator 304, except that the pulse generator 404 includes a primary cell battery 432 for powering the components of the pulse generator 404 instead of a rechargeable battery and a battery charge management chip. The pulse generator 404 further includes a first antenna 422 by which the pulse generator 404 can communicate with the tissue stimulator 106 over a range of 300 MHz to 8 GHz and a second antenna 480 by which the communication module 424 can communicate with the programming module 102 over a higher frequency range of 300 MHz to 8 GHz. Either of the antennas 422, 480 may be a dipole antenna or thin wire antenna.

The pulse generator 404 includes additional components that function substantially similarly to those described for the pulse generator 104. For example, the pulse generator 404 further includes a communication module 424 that relays instructions carried by the signal received from the programming module 102, a controller 426 that processes the instructions to generate a stimulus waveform, a modulator 428 that imparts a frequency in a range of 300 MHz to 8 GHz to the stimulus waveform, an amplifier 430 that imparts the inputted pulse amplitude on the stimulus waveform, and a power detector 460 that can process feedback information received from the tissue stimulator 106.

While the pulse generator 104 has been illustrated as including a single antenna 138 for communicating with a single tissue stimulator 106, in some embodiments, a pulse generator that is otherwise substantially similar in construction and function to the pulse generator 104 may include more than one antenna 138 for communicating respectively with more than one tissue stimulator 106.

Other embodiments of tissue stimulation systems and pulse generators are within the scope of the following claims.

What is claimed is:

1. An implantable pulse generator, comprising:
a controller configured to generate a forward signal carrying electrical energy;
a first antenna configured to send the forward signal to an implanted tissue stimulator such that the implanted tissue stimulator can use the electrical energy to generate one or more electrical pulses and deliver the one or more electrical pulses to a tissue;
a communication module configured to receive instructions carried by an input signal from a programming module for generating the forward signal at the controller; and
a second antenna configured to receive the input signal from the programming module.

2. The implantable pulse generator of claim 1, wherein the implantable pulse generator is a wireless pulse generator.

3. The implantable pulse generator of claim 1, wherein the forward signal is an RF signal.

4. The implantable pulse generator of claim 1, wherein the first antenna is configured to transmit signals having a frequency in a range of 300 MHz to 8 GHz.

5. The implantable pulse generator of claim 1, wherein the first antenna is configured to transmit and receive energy via radiative coupling.

6. The implantable pulse generator of claim 1, wherein the second antenna is configured to transmit signals having a frequency in range of 300 MHz to 8 GHz.

7. The implantable pulse generator of claim 1, wherein the second antenna is configured to transmit and receive energy via inductive coupling.

8. The implantable pulse generator of claim 1, further comprising a rechargeable battery for powering the implantable pulse generator.

9. The implantable pulse generator of claim 6, wherein the second antenna is configured to transmit power to the rechargeable battery.

10. The implantable pulse generator of claim 6, further comprising a third antenna configured to transmit power to the rechargeable battery.

11. The implantable pulse generator of claim 8, wherein the third antenna is configured to transmit signals having a frequency in a range of 300 MHz to 8 GHz.

12. The implantable pulse generator of claim 11, wherein the third antenna is configured to transmit and receive energy via inductive coupling.

13. The implantable pulse generator of claim 1, further comprising a primary cell battery for powering the implantable pulse generator.

14. The implantable pulse generator of claim 1, further comprising one or more additional first antennas for communicating with one or more additional tissue stimulators.

15. The implantable pulse generator of claim 1, further comprising a housing that contains the controller, the first antenna, the second antenna, and the communication module.

16. The implantable pulse generator of claim 1, wherein the housing is hermetically sealed.

17. The implantable pulse generator of claim 1, wherein the housing is not hermetically sealed.

18. The implantable pulse generator of claim 1, further comprising a power detector that can receive a reflected power signal from the implanted tissue stimulator via the first antenna.

19. The implantable pulse generator of claim 18, wherein the controller is configured to adjust the forward signal based on the reflected power signal.

20. The implantable pulse generator of claim 18, wherein the power detector comprises an RF switch.
